(19) United States
(12) Patent Application Publication
(10) Pub. No.: US 2014/0238100 A1
(43) Pub. Date: Aug. 28, 2014

(54) METHOD FOR CALIBRATION OF SENSORS EMBEDDED OR WIRELESSLY CONNECTED TO A MOBILE DEVICE

(71) Applicant: QUALCOMM Incorporated, San Diego, CA (US)

(72) Inventors: Ana Rangelova LONDERGAN, Santa Clara, CA (US); Russel Allyn Martin, Menlo Park, CA (US)

(73) Assignee: QUALCOMM Incorporated, San Diego, CA (US)

(21) Appl. No.: 14/084,456

(22) Filed: Nov. 19, 2013

Related U.S. Application Data
(60) Provisional application No. 61/770,315, filed on Feb. 27, 2013.

(51) Int. Cl. G01N 33/00

(52) U.S. Cl. CPC G01N 33/0006
USPC 73/1.03; 204/406

(57) ABSTRACT

Methods, systems, computer-readable media, and apparatuses for obtaining accurate gas sensor data are presented. A mobile device may receive gas sensor data from at least one gas sensor. The mobile device can include the at least one gas sensor and at least one environmental component. Additionally, the mobile device may receive environmental data from the at least one environmental component. Subsequently, the mobile device may calibrate the received gas sensor data based on the received environmental data and one or more reference characteristic curves. In one or more arrangements, the gas sensor can be a Metal Oxide Semiconductor (MOS) gas sensor or an electrochemical gas sensor configured to sense carbon monoxide.

### CROSS-REFERENCE TO RELATED APPLICATIONS

This application claims priority to Provisional U.S. Application Ser. No. 61/770,315, entitled “SENSOR CALIBRATION,” filed Feb. 27, 2013, which is assigned to the assignee hereof and expressly incorporated herein by reference.

### BACKGROUND

I. Field

The present disclosure relates to computing device technologies. Specifically, various techniques are provided for calibrating one or more chemical sensors in mobile computing devices based on sensor fusion and contextual awareness.

II. Background

Growing number of sensors are being incorporated in smart phones, such as accelerometers, gyroscopes, atmospheric pressure, humidity and temperature sensors. Combined with location assistance data (e.g., indoor positioning unit, Global Positioning System (GPS)) and the high computing power of current mobile devices, sensor fusion and contextual awareness can allow for new development of applications.

Chemical gas sensors may be included in smart mobile devices for monitoring of toxic gases and for monitoring of polluting gases. An example of a toxic gas can include carbon monoxide (CO). Examples of polluting gases can include CO, nitric oxide (NO), nitrogen dioxide (NO₂), ammonia (NH₃), and carbon dioxide (CO₂). In some instances, the two chemical gas sensor applications (i.e., toxic and polluting) have monitoring of CO in common, although the applicable concentration ranges can be different.

Chemical sensors can include electrochemical, conducting polymer, spectroscopic absorption and Metal Oxide Semiconductor (MOS) gas sensors. However, the accuracy of the chemical sensors may be dependent on environmental conditions. Additionally, chemical sensors may need to be frequently calibrated.

In some instances, MOS gas sensors may be favorable for use in smart mobile devices due to the small size and low cost of the sensors. Additionally, the MOS gas sensors can have high sensitivity and fast response times. However, drawbacks can include poor selectivity, dependence on environmental conditions, and need for frequent calibration. Dependence on environment conditions can include temperature dependency and humidity dependency. Additionally, the frequent need of calibration can include complex sensor calibration when high sensor accuracy is required. Furthermore, the MOS gas sensors may have to be heated (e.g., in the range of 100-400 Celsius (° C.)), which can be a drain on the power of the mobile device.

### BRIEF SUMMARY

Various techniques are provided for calibrating sensors embedded or wirelessly connected to a mobile device. Methods, systems, computer-readable media, and apparatuses for obtaining accurate gas sensor data are presented.

Embodiments herein describe how to use information from other sensors on the device (e.g., sensor fusion), environmental information available from the internet for a device location (e.g., based on GPS), and a processor to process the sensor data collected at various conditions, using stored reference data and optimized measurement algorithms to extract accurate chemical sensor readings through the life of the device.

According to some embodiments, inputs from humidity, pressure and/or temperature sensors can be used in addition to the stored calibration curve corresponding to the sensors. Additionally, location assistance data can be used to re-set the zero reading in clean air and calibrate for low pollution levels. Furthermore, the non-linear chemical sensor response as a function of various sensor heater temperatures can be used in specialized data processing algorithms to extract accurate chemical sensor readings. Moreover, two or more sensors with different sensitivity or configuration (e.g., doping, filter, gas pre-concentrator) can be used for increased accuracy.

According to some embodiments, the method may comprise receiving, by a mobile device, gas sensor data from at least one gas sensor. The mobile device can include at least one gas sensor and at least one environmental component. Additionally, the mobile device may receive environmental data from the at least one environmental component. Furthermore, the mobile device can calibrate the received gas sensor data based on the received environmental data and one or more reference characteristic curves.

In one or more arrangements, the gas sensor can be a Metal Oxide Semiconductor (MOS) gas sensor.

In one or more arrangements, the gas sensor can be an electrochemical gas sensor.

In one or more arrangements, the gas sensor can be a polymer-based sensor.

In one or more arrangements, the gas sensor can be a spectroscopic absorption sensor.

In one or more arrangements, the at least one gas sensor can be configured to sense carbon monoxide (CO), nitric oxide (NO), nitrogen dioxide (NO2), ammonia (NH3), or carbon dioxide (CO2).

In some instances, the at least one environmental component includes a positioning unit, and the method may further determine, using the positioning unit, that the mobile device is at an outdoor location. Additionally, the method may receive, using a communications subsystem, clean air data for the outdoor location, wherein the clean air data is associated with an air quality index that indicates a particular quality of air. Furthermore, the method may calibrate the received gas sensor data associated with a zero reading based on the clean air data. For example, the clean air data can be received from an environmental agency or a weather station.

In some instances, the mobile device can include a communications subsystem, and the one or more reference characteristic curves is received from the communications subsystem.

In some instances, the at least one environmental component can include a temperature sensor, and the method may further determine a temperature characteristic curve for the gas sensor data associated with a plurality of temperatures, based on the environmental data from the temperature sensor. Additionally, the calibration of the received gas sensor data can be based on the temperature characteristic curve.

In some instances, the at least one environmental component can include a humidity sensor, and the method may further determine a humidity characteristic curve for the gas sensor data associated with a plurality of humidities, based on the environmental data from the humidity sensor. Additionally, the calibration of the received gas sensor data is based on the humidity characteristic curve.

In some instances, the at least one environmental component can include an atmospheric pressure sensor, and the method can further determine an atmospheric pressure characteristic curve for the gas sensor data associated with a plurality of atmospheric pressures, based on the environmental data from the atmospheric pressure sensor. Additionally, the calibration of the received gas sensor data is based on the atmospheric pressure characteristic curve.

In some instances, the calibration can be based on a sensor noise level associated with the at least one gas sensor.

In one or more arrangements, the mobile component(s) can include a heater. Additionally, the calibration may further comprise: cooling down the at least one gas sensor by turning off the heater; determining a cooling down gas sensor resistance characteristic curve for the gas sensor data associated with a plurality of temperatures as the at least one gas sensor is cooling down; and calibrating the received gas sensor data based on the cooling down gas sensor resistance characteristic curve.

In one or more arrangements, the mobile component(s) include a first gas sensor and a second gas sensor attached to a filter.

In one or more arrangements, the mobile component(s) include a first gas sensor and a second gas sensor attached to a gas pre-concentrator.

In one or more arrangements, the mobile component(s) include a first gas sensor and a second gas sensor with a different sensitivity to sensing carbon monoxide, and wherein the different sensitivity of the second gas sensor is due to, for example, doping the second gas sensor.

According to some embodiments, a mobile device for obtaining accurate gas sensor data may comprise: memory; at least one gas sensor configured to generate gas sensor data; at least one environmental component configured to generate environmental data; and one or more processors configured to calibrate the generated gas sensor data based on the generated environmental data and one or more reference characteristic curves.

According to some embodiments, one or more computer-readable media storing computer-executable instructions for obtaining accurate gas sensor data that, when executed, may cause one or more computing devices included in a mobile device to: receive gas sensor data from at least one gas sensor, wherein the mobile device includes the at least one gas sensor and at least one environmental component; environmental data from the at least one environmental component; and calibrate the received gas sensor data based on the received environmental data and one or more reference characteristic curves.

According to some embodiments, an apparatus for obtaining accurate gas sensor data may comprise: a means for receiving gas sensor data from at least one gas sensor; a means for receiving environmental data from at least one sensor component; and a means for calibrating the received gas sensor data based on the received environmental data and one or more reference characteristic curves.

### BRIEF DESCRIPTION OF THE DRAWINGS

Aspects of the disclosure are illustrated by way of example. The following description is provided with reference to the drawings, where like reference numerals are used to refer to like elements throughout. While various details of one or more techniques are described herein, other techniques are also possible. In some instances, well-known structures and devices are shown in block diagram form in order to facilitate describing various techniques.

A further understanding of the nature and advantages of examples provided by the disclosure can be realized by reference to the remaining portions of the specification and the drawings, wherein like reference numerals are used throughout the several drawings to refer to similar components. In some instances, a sub-label is associated with a reference numeral to denote one of multiple similar components. When reference is made to a reference numeral without specification to an existing sub-label, the reference numeral refers to all such similar components.

FIG. 1 illustrates an example device that may implement one or more aspects of the disclosure.

FIG. 2 illustrates another example device that may implement one or more aspects of the disclosure.

FIG. 3 illustrates a flowchart of an exemplary method for obtaining accurate gas sensor data, according to some embodiments.

FIG. 4 is an example from a typical sensor response (e.g., sensor resistance) in relations to gas concentration, according to some embodiments.

FIGS. 5A-F illustrate methods of calibrating the received gas sensor data based on the received environmental data, according to some embodiments.

FIG. 6 illustrates an example of calibration module with different software modules for executing the tasks associated with embodiments of the present invention.

FIGS. 7A-C illustrate examples of using two or more sensors for the same target gas, according to some embodiments.

FIG. 8 illustrates some characteristics of the moisture effect on an exemplary sensor with a filter, according to some embodiments.

FIG. 9 shows an example of the temperature profile of a heater in a gas sensor designed for fast heating and cooling, according to some embodiments.

FIG. 10 illustrates a computer device, which may implement, perform, and/or execute some of the embodiments of the present invention.

### DETAILED DESCRIPTION

Several illustrative embodiments will now be described with respect to the accompanying drawings, which form a part hereof. While particular embodiments, in which one or more aspects of the disclosure may be implemented, are described below, other embodiments may be used and various modifications may be made without departing from the scope of the disclosure.

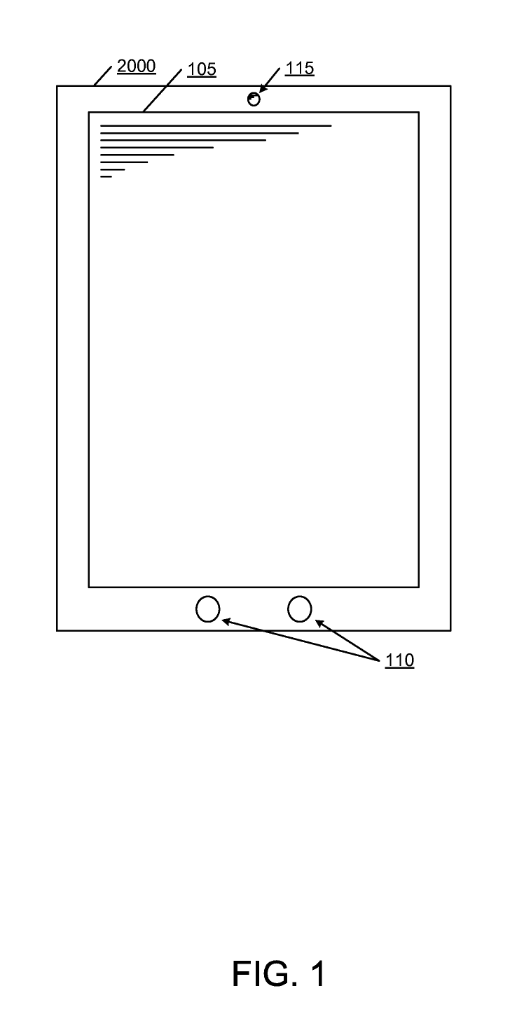

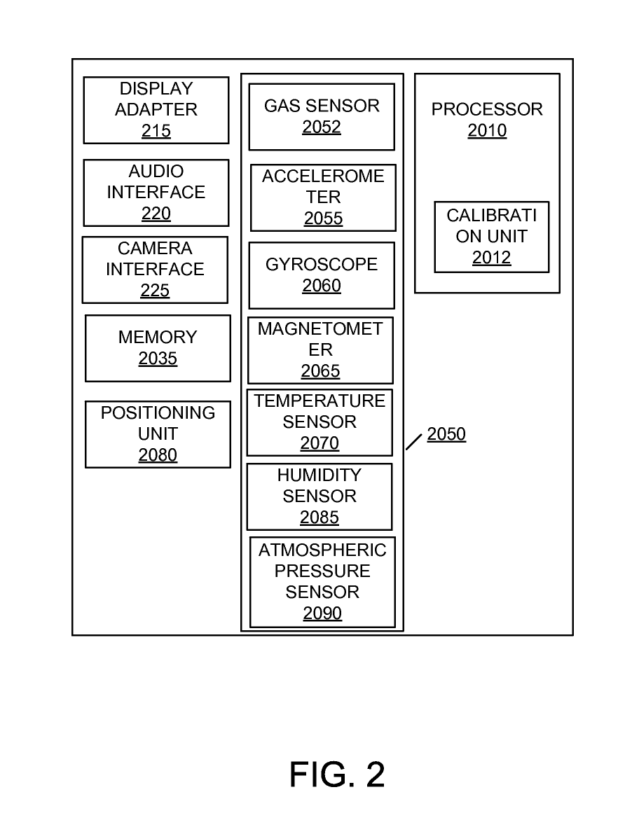

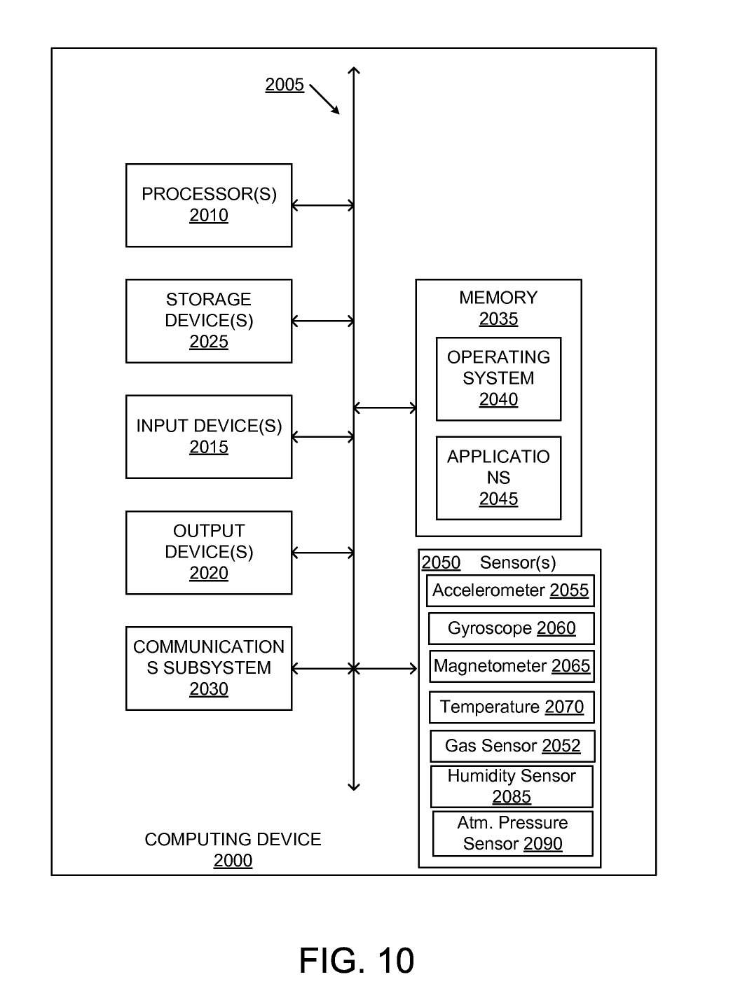

FIGS. 1 and 2 illustrate example devices that may implement one or more aspects of the disclosure and may implement components of the computing device 2000 described in more detail with reference to FIG. 10.

As illustrated in FIG. 1, for example, computing device 2000 may include one or more components such as a display 105, buttons and/or keys 110, and/or a camera 115. In some arrangements, display 105 may be a touch screen, such that a user may be able to provide touch-based user input to computing device 2000 via display 105.

In one or more arrangements, computing device 2000 may further include a plurality of internal components, such as those illustrated in FIG. 2. For example, computing device 2000 may include one or more processors (e.g., processor 2010), one or more memory units (e.g., memory 2035), at least one display adapter (e.g., display adapter 215), at least one audio interface (e.g., audio interface 220), one or more camera interfaces (e.g., camera interface 225), and/or other components. Further, the computing device 2000 may comprise one or more positioning units 2080 configured to determine a position of the computing device 2000. For example, the one or more positioning units can use a satellite positioning system (SPS) such as GPS, or using any other method such as wireless area network (WAN) or wireless local area network (WLAN) positioning, trilateration, triangulation, etc. In some embodiments, the processor 2010 is a CPU and/or an application processor. In some embodiments, the processor 2010 is a sensors processor or core that is separated from a CPU or application processor. Other embodiments may be implemented as well.

In one or more embodiments, computing device 2000 may further include one or more sensors 2050. For example, one or more gas sensors (e.g., gas sensor 2052, described in further detail below), one or more accelerometers (e.g., accelerometer 2055), one or more gyroscopes (e.g., gyroscope 2060), one or more magnetometers (e.g., magnetometer 2065), one or more temperature sensors (e.g., temperature sensor 2070), one or more humidity sensors (e.g., humidity sensor 2085), one or more atmospheric sensors (e.g., atmospheric pressure sensor 2090) and/or one or more other sensors may be included in the computing device 2000 or may be in communication with the computing device 2000 such that the computing device 2000 may receive readings from that sensor even if that sensor is not integrated into the computing device 2000.

Accelerometer 2055 may measure and/or provide information (e.g., to processor 2010) about acceleration experienced by computing device 2000.

Gyroscope 2060 may similarly measure and/or provide (e.g., to processor 2010) information regarding an orientation of computing device 2000.

Magnetometer 2065 may measure and/or provide (e.g., to processor 2010) information regarding a bearing of computing device 2000.

Temperature sensor 2070 may measure and/or provide (e.g., to processor 2010) information about a temperature (e.g., an ambient temperature) experienced by computing device 2000.

Humidity sensor 2085 may measure and/or provide (e.g., to processor 2010) information about a humidity (e.g., moisture content in the air) experienced by computing device 2000. Examples of humidity sensors can include, but are not limited to, hygrometers, capacitive humidity sensors, resistive humidity sensors, chilled mirror dewpoint hygrometers.

Atmospheric pressure sensor 2090 may measure and/or provide (e.g., to processor 2010) information about the pressure (e.g., atmospheric pressure) experienced by computing device 2000. An example of an atmospheric pressure sensor 2090 can include, but not be limited to, a digital barometric pressure sensor.

Although a dotted box is shown enclosing the sensors 2050, these sensors may be implemented separately.

According to one or more aspects, any and/or all of the sensors included in computing device 2000 may generate an electric signal in response to and/or as an output signal corresponding to changes in condition, such as motion detected by a particular sensor when the sensor and/or computing device 2000 are moved in one or more directions and/or gases or chemicals detected by the sensor.

Additionally, or alternatively, because of imperfections in how such sensors may be manufactured, normal deterioration during expected operation, and/or other factors that may create and/or contribute to inaccuracy in the data output by such sensors over time, it may be desirable to calibrate any and/or all of the sensors included in computing device 2000 so as to more accurately interpret signals output by such sensors.

As used herein, “calibrating” a sensor may include receiving raw sensor measurement data (e.g., an electrical signal) output by a sensor, such as a gas sensor, accelerometer, gyroscope, magnetometer, etc. and applying one or more scale factors and/or offsets and/or choosing a calibration curve to the raw data to calculate and/or otherwise determine a more accurate representation of motion corresponding to the received sensor data. The scale factors and/or offsets and/or calibration curves may independently and/or together be referred to as “calibration parameters,” and each sensor may have its own corresponding set of calibration parameters.

In addition, in applying calibration parameters to sensor data, a “scale factor” may be a coefficient or other value that is multiplied by received sensor data, and an “offset” may be a value that is added to the received sensor data. In some embodiments, the processor 2010 includes a calibration unit 2012 configured to calibrate one or more of the sensors 2050. In some embodiments, there may be a calibration unit for each sensor. In some embodiments, the calibration unit 2012 is implemented separately from the processor 2010. In some embodiments, the calibration unit 2012 may be implemented in one or more of the sensors 2050.

In many current systems, calibration of sensors may be done at the time a computing device 2000 is manufactured (e.g., at the factory). In addition, or instead, calibration may be performed during operation of the computing device 2000 or otherwise subsequent to manufacture. In some embodiments, information from one or more of the sensors 2050 may be combined with information from the positioning unit(s) 2080 to open novel application opportunities through sensor fusion and contextual awareness.

The chemical gas sensor can be implemented by the gas sensor 2052. Example of the chemical gas sensor can include an electrochemical, a conducting polymer, a spectroscopic absorption and a Metal Oxide Semiconductor (MOS) gas sensor. Furthermore, the chemical gas sensor may be included in computing device 2000 such as a mobile device. Such gas sensor may be used to monitor for toxic gases (e.g., CO) for personal safety, and environmental monitoring for polluting gases (e.g., CO, NO, NO₂, NH₃, CO₂). Both of these applications (i.e., monitoring of toxic and polluting gases) have monitoring of CO in common, although the applicable concentration ranges are different. As shown below in Table 1, a CO sensor range of 1-500 ppm may cover both and such sensor may be implemented in gas sensor 2052. Table 1 illustrates the environmental range for CO based on Environmental Protection Agency (EPA) data.

TABLE 1
Environmental Range for CO - EPA Table.

| | | “Good” Level | “Moderate” Level | “Hazardous” |
|---|---|---|---|---|
| GAS | Units | (50) | (100) | Level (500) |
| Carbon | ppm | 4.4 | 9.4 | 50.4 |
| Monoxide | | | | |
| (8 Hr Avg) | | | | |

In one or more arrangements, the gas sensor 2052 can be a MOS gas sensor The MOS gas sensor can be used in a mobile device because of its small size, low cost in volume manufacturing, high sensitivity and fast response times. Such properties may be beneficial in certain implementations when integrated into or associated with a mobile device. Certain drawbacks, however, may be present, which may include poor selectivity, dependence on environmental conditions (e.g., temperature, humidity), and frequent, complex sensor calibration if sensor accuracy is required.

Furthermore, the chemical gas sensor may have to be heated (e.g., in the range of 100-400 Celsius (° C.)), which can be a drain on the power of the mobile device. However, low power consumption (e.g., about 1-2 mW on average) can be achieved through sensor miniaturization and optimized sequence for turning the heater on for a short duration (e.g., few tenths of a sec) only when taking a reading.

In another arrangement, an electrochemical sensor can be implemented in gas sensor 2052. The electrochemical sensor may be used due to the good accuracy, sensitivity, selectivity and product maturity. In some instances, the electrochemical sensor may be bulky (e.g., more than 10 mm thick), and may not be packaged inside a thin form factor mobile device. However, as electrochemical sensors shrink in size, the electrochemical sensors can be included in a mobile device. Subsequently, the mobile device can use an electrochemical sensor to monitor toxic and polluting gases. Therefore, the electrochemical sensor may need to be calibrated in a similar manner to the other chemical sensors, with the exception of the features related to the non-linear behavior of MOS gas sensors.

Additionally, sending a mobile device back to the factory for re-calibration or doing a conventional sensor re-calibration for a particular gas using the particular gas sample standards is inconvenient for a typical smart phone user.

According to some embodiments, the chemical gas sensor can be frequently calibrated, in order to obtain accurate sensor readings (e.g., within 5%) throughout the life of a mobile device (e.g., more than three years). The calibration can be based on: information from other sensors on the phone; environmental information received from the internet; and processing the gas sensor data using stored reference data (e.g., based on characteristic curve) and optimized measurement algorithms.

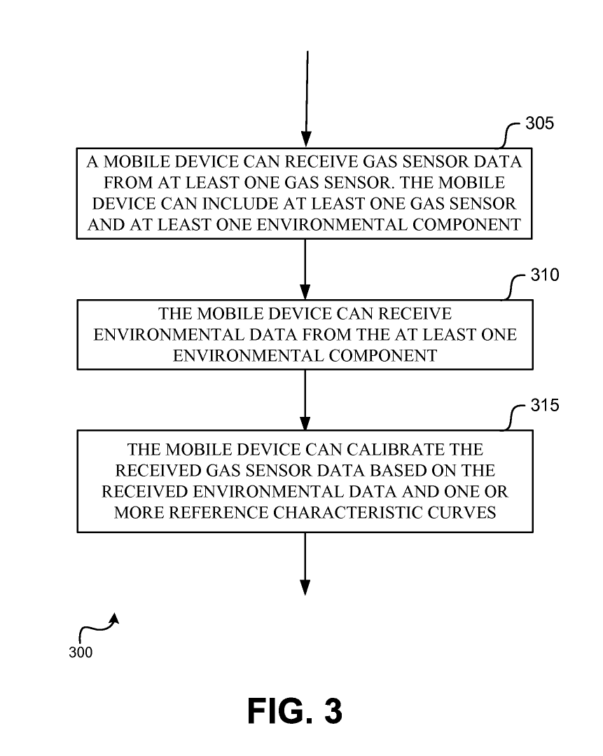

FIG. 3 illustrates a flowchart of an exemplary method 300 for obtaining accurate gas sensor data, according to some embodiments. For example, a mobile device can be configured to obtain environmental data based on an environment of the mobile device. Additionally, to get more accurate readings, the mobile device can process the sensor data using the received environmental data and reference characteristic curve(s). In some instances, the reference data can be stored in the memory of the mobile device. Alternatively, the reference characteristic curve(s) can be received in real-time via the internet. In one or more arrangements, reference characteristic curve(s) can include detailed characteristic curves for sensor response under various temperatures, atmospheric pressure and humidity conditions.

At 305, a mobile device can receive gas sensor data from at least one gas sensor. The mobile device can include at least one gas sensor and at least one environmental component. The gas sensor(s) 2052 can include, but not limited to, a MOS gas sensor, an electrochemical sensor, a polymer-based sensor, a gas sensor for sensing CO, and any other sensors for sensing a toxic or polluting gas. The mobile component(s) can be, but is not limited to, an accelerometer 2055, a gyroscope 2060, a magnetometer 2065, a temperature sensor 2070, a positioning unit(s) 2080, a humidity sensor 2085, and an atmospheric pressure sensor 2090.

In one or more arrangements, the mobile device (e.g., computing device 2000) at 305 can receive gas sensor data from a gas sensor(s) 2052 using a bus 2005. In such arrangement, at least portions of 305 may be performed, for example, by the processor 2010 in combination with information and/or instructions from memory 2035.

At 310, the mobile device can receive environmental data from the at least one environmental component. In one or more arrangements, the mobile device (e.g., computing device 2000) at 310 can receive environmental data using a communications subsystem 2030 and/or sensor(s) 2050 using a bus 2005. Sensor(s) 2050 can include an accelerometer 2055, a gyroscope 2060, a magnetometer 2065, a temperature sensor 2070, a humidity sensor 2085 and/or an atmospheric pressure sensor 2090. In such arrangement, at least portions of 310 may be performed, for example, by the processor 2010 in combination with information and/or instructions from memory 2035.

For example, in one or more arrangements, the mobile device can use environmental data from an environmental sensor (e.g., sensor(s) 2050) to compensate for environmental effects (e.g., humidity, temperature, atmospheric pressure) on gas sensor readings from a gas sensor 2052.

In some instances, the effect of surrounding environment can be more significant when there is no filter on the gas sensor, which may be the case for a gas sensor embedded in a mobile device. However, in one or more arrangements, a small size filter which can fit inside the mobile device can be used with the gas sensor to lessen the environmental effects.

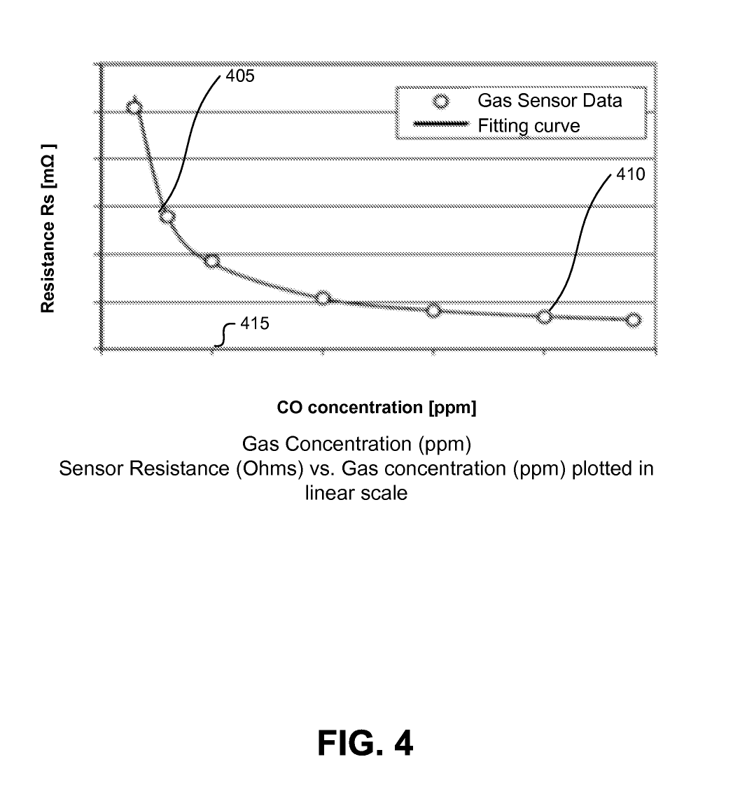

At 315, the mobile device can calibrate the received gas sensor data based on the received environmental data and one or more reference characteristic curves. In such arrangement, at least portions of 315 may be performed, for example, by the processor 2010 in combination with information and/or instructions from memory 2035. FIGS. 4A-F illustrate examples of calibrating the received gas sensor data based on the received environmental data and one or more reference characteristic curves.

As it is known in the art, sensor response may not be a linear function of gas concentration. Therefore, a minimum of three data points collected for three different concentrations of the specific gas can be received and used for calibration. Four points may be used for improved accuracy in some embodiments, for example by the calibration unit 2012.

FIG. 4 is an example from a typical sensor response (e.g., sensor resistance) in relations to gas concentration, according to some embodiments using a metal oxide sensor.

In some embodiments, the techniques may be used to calibrate a gas sensor, for example with the calibration unit 2012 when the sensor is implemented in the gas sensor 2052.

As illustrated in FIG. 4, the sensor response to the gas concentration is non-linear showing large changes of resistance (Rs) at low concentration and a saturating behavior towards higher concentrations of CO as shown by the measured points of the gas sensor.

In some embodiments, the calibration unit 2012 can use the characteristic curve (e.g., fitting calibration curve) to determine the CO concentration detected by the gas sensor 2052. The fundamental dependency of the gas sensor 2052 can be described as an inverse function as follows.

Rs=a+[b/(CO level)] Equation 1

In equation 1, ‘a’ is the resistance at an infinite level of CO and ‘b’ describes resistance variation due to CO exposure.

Additionally, from equation 1, the CO level can then be calculated with the function as follows.

CO level=b/(Rs−a) Equation. 2

Based on equations 1 and 2, ‘b’ can be derived by measuring the gas sensor 2052 responses at two or more points (e.g., point 405, point 410). Using the derived equation, the calibration unit 2012 can calculate the CO level over a defined range with a low error (e.g., less than 5%).

In one or more arrangements, the sensor noise level can be used to identify the various regions of gas concentration. As previously mentioned, FIG. 4 illustrates a plot of the gas sensor 2052 resistance level as a function of gas concentration in a linear scale, according to one embodiment. The sensor noise and readings variation with very small CO concentration changes will be significantly larger in the low parts-per-million (PPM) range. Therefore, using and characterizing the sensor noise level, the mobile device can improve the sensor accuracy.

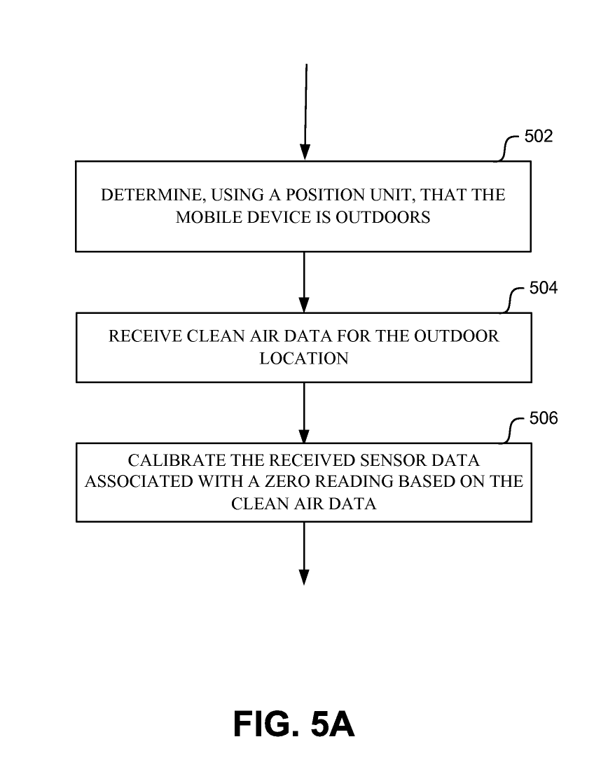

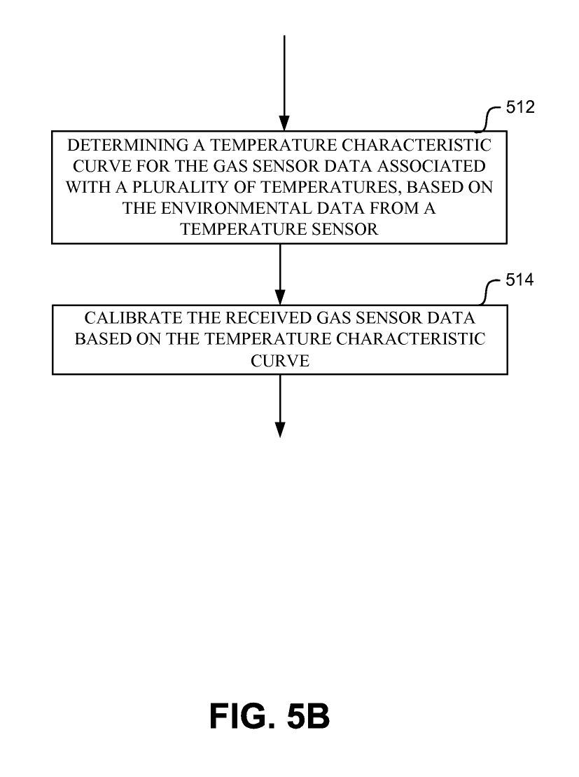

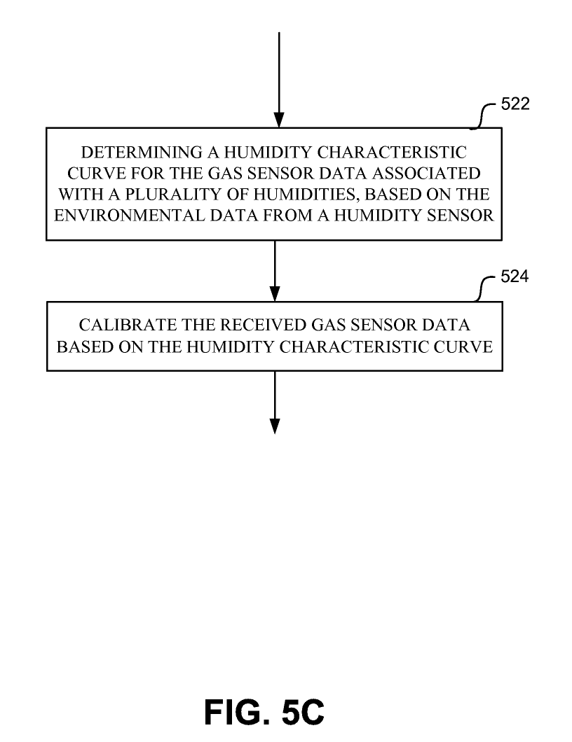

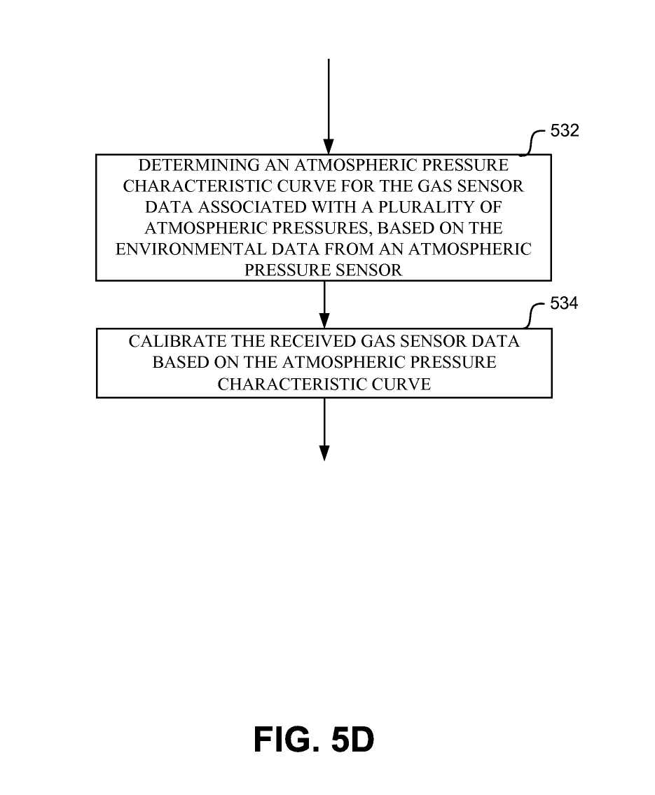

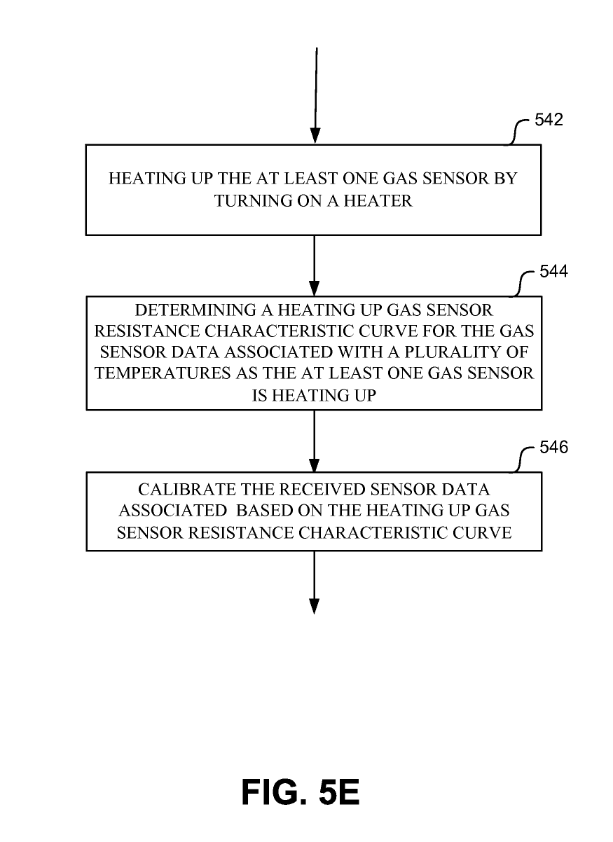

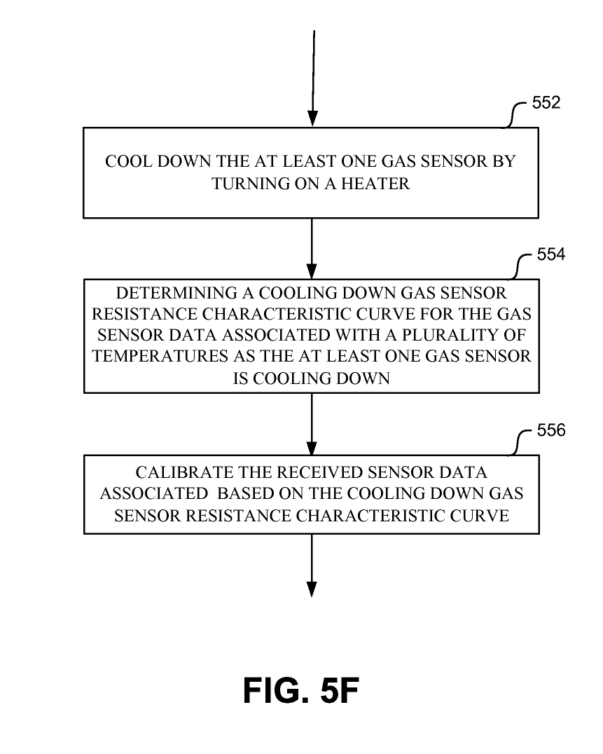

Now referring to FIG. 5A, which illustrates a method of calibrating the received gas sensor data based on the received environmental data (e.g., clean air data) using a positioning unit 2080.

At 502, the mobile device can determine, using the positioning unit(s) 2080, that the mobile device is outdoors. For example, in one or more arrangements, the gas sensor data can be tracked over time and the re-adjusted to a zero reading as necessary when the mobile device is outside in open air as determined by the positioning unit (e.g., GPS).

At 504, the mobile device can receive clean air data from the outdoor location using communications subsystem 2030. The zero reading can be compared to readings from the mobile device, when the mobile device is outdoors in a clean air environment. The clean air environment classification can be based on the Environmental Protection Agency (EPA) classification and data. For example, the EPA air quality data, which can be updated online on an hourly-basis, can be downloaded by the mobile device based on the location of the mobile device. In some instances, the location of the mobile device can be determined based on GPS data.

At 506, the mobile device can calibrate the received sensor data associated with a zero reading based on the clean air data. In some instances, the concentration of CO from the sensor readings for an environment with low air pollution can become a data point in the low range of the sensors curve associated with concentration versus output level. Furthermore, multiple samples may be taken within a narrow time frame to remove any outliers. For example, occasional peaks may be ignored because the peaks may correspond to areas of higher local pollution.

According to some embodiments, accurate gas concentration can be determined by: applying stored calibration coefficients; calibrating using high/low CO pollution days; processing at various temperatures; using the sensor noise level. For example, the mobile device can apply stored calibration coefficients to the measured response to a gas at the optimal heater temperature to get a rough number for the concentration.

In one or more arrangements, the sensor can be calibrated using high CO pollution days to provide points on the calibration curve. The calibration of the sensors in the low CO concentration range can be done in cities where the pollution values may be lower, using EPA or other similar information that is available on the web. For example, by using geofencing and location data, the mobile device can better calibrate the sensors. Additionally, once the sensor is calibrated, the sensor can be used in areas where local CO data is not available, in localized higher pollution areas (e.g., sitting in traffic), or when there is no internet connection (e.g., a remote cabin in the mountains).

The calibration of the gas sensor 2052 in the low CO concentration range can be done, for example by the calibration unit 2012, in cities where the pollution values may be higher, using EPA or other similar information that is available on the internet or from another source.

FIG. 5B illustrates a method of calibrating the received gas sensor data based on the received environmental data from a temperature sensor 2070. Environmental data from the temperature sensor 2070 can include the temperature reading of the environment around the mobile device. Given that gas concentration is correlated to the environmental temperature, a more accurate gas reading can be determined by calibrating the raw gas sensor data based on the environmental data received from the temperature sensor 2070.

In some embodiments, input from the temperature sensor 2070 can be used to compensate for temperature variation. In some instances, the gas temperature in the sensor headspace can be used to calibrate the raw sensor reading from the gas sensor 2052.

At 512, the mobile device can determine a temperature characteristic curve for the gas sensor data associated with a plurality of temperatures, based on the environmental data from the temperature sensor 2070.

At 514, the mobile device can calibrate the received gas sensor data based on the temperature characteristic curve.

Additionally, the plot corresponding to the gas concentration in relations to temperature can be unique for each gas concentration and humidity conditions. Furthermore, the effects of the humidity conditions can be compensated based on adjustments due to readings from an independent humidity sensor.

FIG. 5C illustrates a method of calibrating the received gas sensor data based on the received environmental data from a humidity sensor 2085.

At 522, the mobile device can determine a humidity characteristic curve for the gas sensor data associated with a plurality of humidities, based on the environmental data from the humidity sensor 2085.

At 524, the mobile device can calibrate the received gas sensor data based on the humidity characteristic curve.

In some embodiments, input from a humidity sensor 2085 (e.g., relative humidity sensor) can be used to compensate for humidity effects on gas sensor 2052 response. This can depend on the filter and specific sensor design.

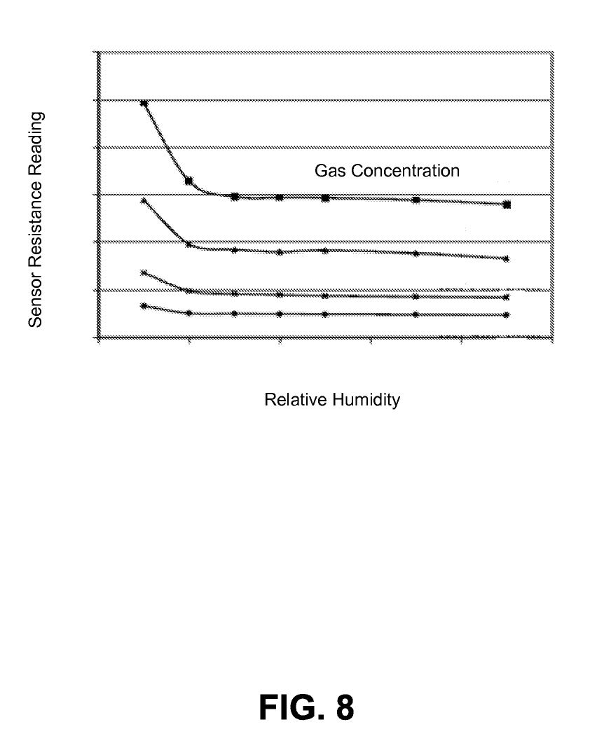

FIG. 8 illustrates some characteristics of the moisture effect on an exemplary CO sensor (e.g., gas sensor 2052) with a filter. As illustrated in FIG. 8, in some instances, the sensor output does not change much in a typical 20 to 95% relative humidity. Additionally, the effect of humidity is more significant when the sensor does not have a filter, or only has a small size filter, which may be the case for a sensor embedded in a mobile device where space is limited. In a case with no filter, humidity compensation may be necessary and important for accuracy.

According to another embodiment, the gas concentration sensor reading (e.g., CO concentration) can be dependent on the atmospheric pressure.

FIG. 5D illustrates a method of calibrating the received gas sensor data based on the received reference data from an atmospheric pressure sensor 2090.

At 532, the mobile device can determine an atmospheric pressure characteristic curve for the gas sensor data associated with a plurality of atmospheric pressures, based on the environmental data from the atmospheric pressure sensor 2090.

At 534, the mobile device can calibrate the received gas sensor data based on the atmospheric pressure characteristic curve.

In some instances, a chemical gas sensor can work as an electrical transducer that converts the presence of gas into a measurable signal. For example, a MOS gas sensor can have a metal oxide-based sensing layer deposited onto a substrate. The substrate can contain electrodes that measure the resistance of the sensing layer and a heater (not pictured in FIG. 2) that heats up the sensing layer. At high enough temperatures (e.g., 100-400° C.), many gases can interact with the sensing layer (e.g., surface of the metal oxide), which can cause the resistance of the sensing layer to change. The MOS gas sensor can respond to changes in the composition of the ambient atmosphere with a change in the resistance of the sensing layer.

Accordingly, the data for the plot of the gas concentration in relations to temperature can be collected at various heater ramp rates, as the shape of the curve associated with the plot can differ depending on the time the gas has to interact with the sensor element at various temperature points. For example, a family of these curves and plots can be collected within a few minutes and used to further improve the accuracy of the gas concentration measurements.

In one or more arrangements, the sensors can be calibrated by processing the entire response of the gas sensor at various temperatures as the heater is ramped up to the optimal temperature to refine the result. As previously mentioned, the sensor resistance, and hence gas readings, are a function of the heater temperature and the gas associated with the sensor. In some instances, the mobile device can record the sensor resistance just before the heater is turned on and during the heat up period (e.g., 6-7 sec).

FIG. 5E illustrates a method of calibrating the received gas sensor data based on the received reference data (e.g., heating up ramp rates).

At 542, the mobile device can heat up the gas sensor(s) 2052 by turning on a heater.

At 544, the mobile device can determine a heating up gas sensor resistance characteristic curve for the gas sensor data associated with a temperatures as the gas sensor(s) 2052 is heating up.

At 546, the mobile device can calibrate the received gas sensor data based on the heating up gas sensor resistance characteristic curve.

For example, the mobile device can process the entire response of the gas sensor 2052 at various temperatures, using calibration unit 2012, as the heater is ramped up to the optimal temperature to refine the result.

In a low power scenario where the heater is turned on only before and during the measurement, the heater temperature may be allowed to stabilize before recording the gas sensor 2052 element resistance.

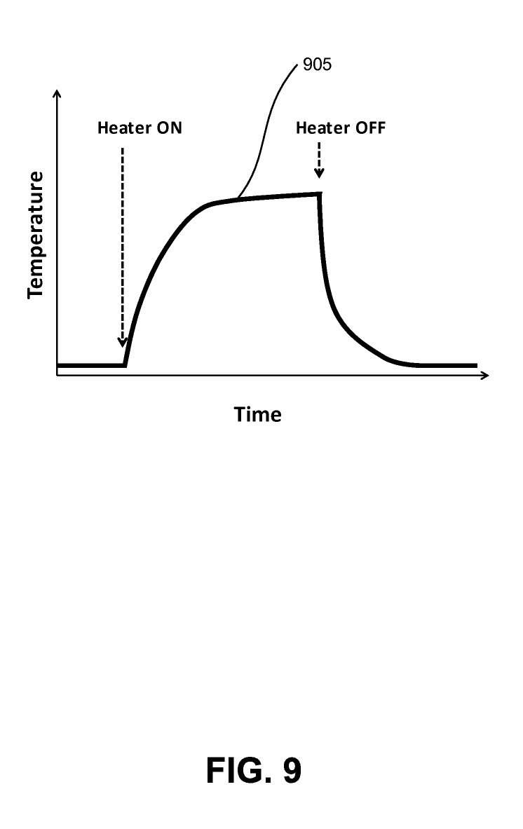

FIG. 9 shows an example of the temperature profile of a heater in a gas sensor designed for fast heating and cooling, according to some embodiments. As illustrated at 905, 90% of the 500° C. set point is reached in 0.3 sec, after which a measurement can be taken. Therefore, according to some embodiments, the operation temperature can be carefully selected for optimal selectivity for the target gas and low power consumption. For example, an operation temperature for gas (e.g., CO) monitoring may be in the 120° C. to 150° C. range.

Alternatively, curves and plots for cooling down the gas sensor(s) 2052 can be used to further improve the accuracy of the gas concentration measurements.

FIG. 5F illustrates a method of calibrating the received gas sensor data based on the received reference data (e.g., cooling down ramp rates).

At 552, the mobile device can cool down the gas sensor(s) 2052 by turning off the heater.

At 554, the mobile device can determine a cooling down gas sensor resistance characteristic curve for the gas sensor data associated with a plurality of temperatures as the gas sensor(s) 2052 is cooling down.

At 556, the mobile device can calibrate the received gas sensor data based on the cooling down gas sensor resistance characteristic curve.

Embodiments described herein focused on chemical gas sensors. According to some embodiments of the present invention, the teachings herein can be applied to any chemical gas sensors (e.g., MOS, electrochemical, polymer-based, spectroscopic absorption).

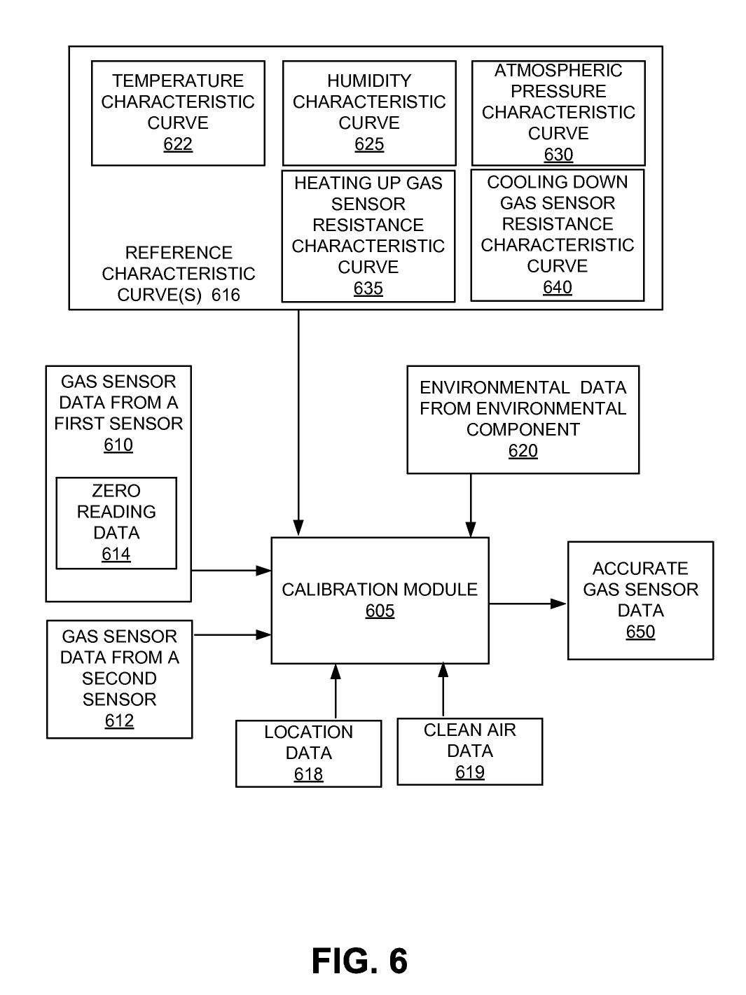

Now referring to FIG. 6, which illustrates an example of calibration module 605 with different software modules for executing the tasks associated with embodiments of the present invention. In some arrangements, the instructions/code for carrying the tasks associated with the different software modules can be stored in the memory 2035 of the mobile device. In some arrangements, the instructions/code can be executed by processor 2010 and calibration unit 2012 of the mobile device.

In one or more arrangements, the calibration module 605 can receive gas sensor data 610 from a first sensor. For example, the gas sensor 2052 can be an example of the first sensor. Additionally, the calibration module 605 can receive environmental data 620 from an environmental component. For example, the temperature sensor 2070, the humidity sensor 2085, the atmospheric pressure sensor 2090 can be examples of the environmental component. Furthermore, the calibration module 605 can receive one or more reference characteristic curve(s) 616. The calibration module can calibrate the received gas sensor data based on the received environmental data and the one or more reference characteristic curve(s) to output accurate gas sensor data 650. Method 300 in FIG. 3 illustrates an example according to this embodiment in more details.

Additionally, in some instances, sensor re-calibration may comprise a two-step process. First, a zero reading reset, if a zero shift is detected. Secondly, a span correction can be used in order for the output to reflect a correct measured value. As previously mentioned, some chemical gas sensors can detect gas concentration by measuring the increase in conductivity (i.e., drop of resistance) across the sensing element.

When a zero reading reset is required, the calibration module 605 can receive location data 618, zero reading data 614 and clean air data 619 to re-calibrate the sensor readings. For example, the location data 618 can be received from positioning unit(s) 2080. Additionally, the clean air data 619 can be received from a third party, such as, but not limited to, the EPA, and a weather station. Using the clean air data 619, the calibration module 605 can determine that the outdoor air is clean (e.g., based on EPA standards). Furthermore, the zero reading data 614 can be included in the gas sensor data 610 from the first sensor when the mobile device is outdoors and the clean air data 619 implies that the outdoor air is clean. FIG. 5A discusses an example according to this embodiment in more details.

In some embodiments, the zero reading of gas sensor 2052 can be compared to readings when the mobile device determines that the outdoor air is classified as clean air. According to EPA data, the average CO concentration in the air in the USA is less than 0.5 ppm. The sensor readings can be tracked over time and the zero re-adjusted as necessary when the user is, for example in the park or area free of CO sources as determined by the positioning unit(s) 2080. The EPA air quality data, which can be updated hourly, can be downloaded to the mobile device based on the location of the mobile device. In some instances, the zero re-set will proceed if the air quality is shown as “green” or good by the EPA color code for that location. Several measurements can be made by the gas sensor 2052 and spikes due to localized sources can be removed, for example by the calibration unit 2012.

In one or more arrangements, the one or more reference characteristic curve(s) 616 received by calibration module 605 can include, but is not limited to, a temperature characteristic curve 622, a humidity characteristic curve 625, an atmospheric pressure characteristic curve 630, a heating up gas sensor resistance characteristic curve 635, and a cooling down gas sensor resistance characteristic curve 640. FIGS. 5B-F discuss examples according to these embodiments in more details.

For example, detailed characteristic curves (e.g., temperature characteristic curve 622, humidity characteristic curve 625, atmospheric pressure characteristic curve 630, heating up gas sensor resistance characteristic curve 635, cooling down gas sensor resistance characteristic curve) for sensor response under various temperature, atmospheric pressure and humidity conditions are stored in the memory 2035.

In one or more arrangements, the calibration module 605 can receive gas sensor data from a second sensor 612. For example, the gas sensor data from the second sensor 612 can be in addition to the received gas sensor data 610 from the first sensor.

According to some embodiments, the accuracy of the measurement can be improved by using two or more sensors for the same target gas. These sensors can be engineered to have different sensitivity to the target gas. Processing the response from two or more sensors will therefore dramatically improve the accuracy.

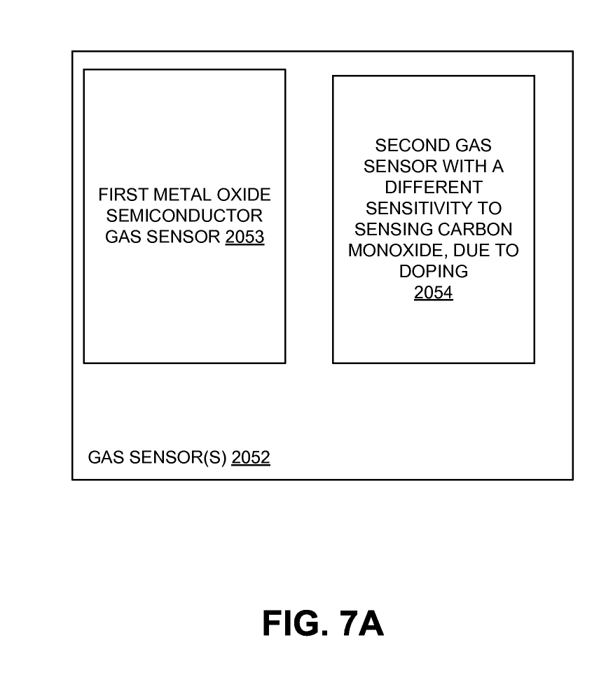

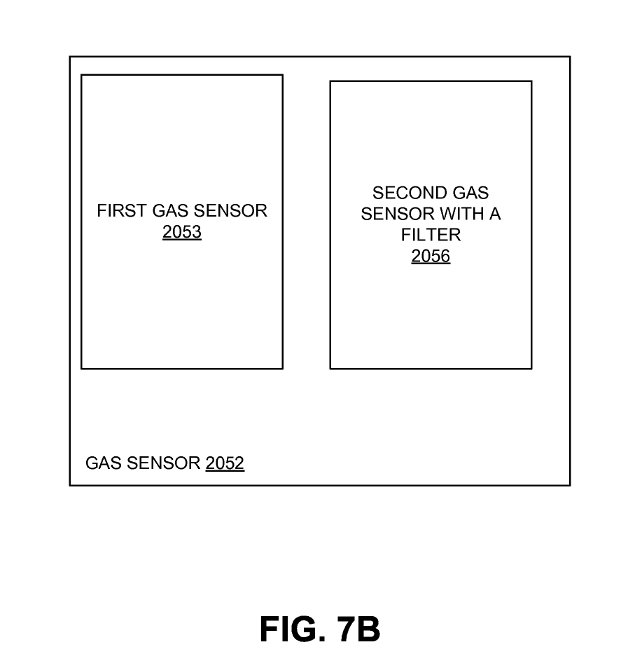

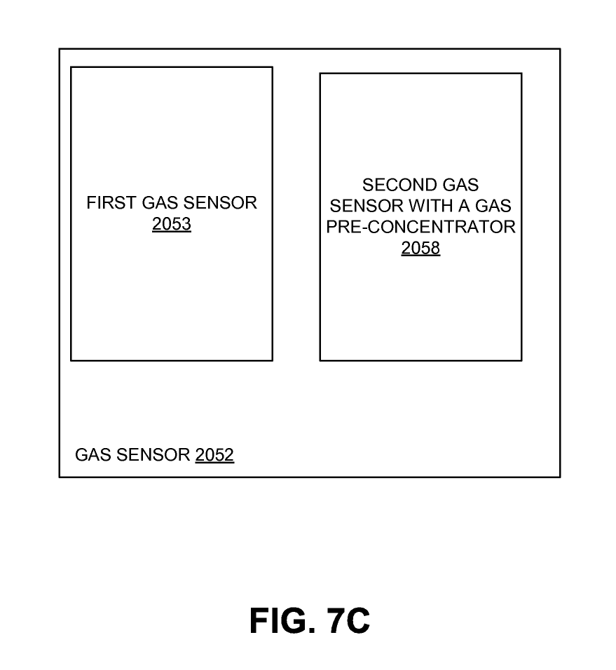

FIGS. 7A-C illustrate examples of using two or more sensors for the same target gas.

In some instances, since the sensor size is much smaller compared to the final package size, multiple sensor elements can be included with only a small (i.e., about a 10 to 20%) increase in the physical size of the packaged sensor part.

One approach to change the sensors sensitivity is by doping the sensor element. For example, a gas sensor based on tin oxide (SnO) can be doped with, for example, platinum (Pt) or palladium (Pd). According to one embodiment, FIG. 7A illustrates the gas sensor(s) 2052 with a first gas sensor 2053 and a second gas sensor 2054 with a different sensitivity to sensing a specific gas due to doping. In some instances, processing the response from two or more sensors, for example by the calibration unit 2012, may therefore dramatically improve the accuracy.

Another approach is it to place a small filter in front of one sensor and not use a filter with the other. The readings of the sensor will be correlated to the specific filter, especially in the transient temperature region after the heater is powered up. According to one embodiment, FIG. 7B illustrates the gas sensor(s) 2052 with a first gas sensor 2053 and a second gas sensor 2056 with a filter.

In some embodiments, a small filter is placed in front of the second gas sensor 2056 and a filter is not used with the first gas sensor 2053. The readings of the second gas sensor 2056 may be a strong function of the specific filter, especially in the transient temperature region after the heater is powered up. Provisions may be taken for periodical de-gassing of the filter. In one example, de-gassing can be achieved by heating it to a set temperature.

In yet another approach, a gas pre-concentrator can be placed in front of one sensor and the readings compared to the readings of a sensor without pre-concentrator. Using two sensors can provide two different concentration points for the same ambient exposure level. The difference between the two readings can also be used to indicate the approximate gas concentration. For example, the difference may be larger at smaller concentrations (e.g., less than 100 ppm CO concentration) and smaller at larger concentrations (e.g., more than 100 ppm CO concentration).

Additionally, the use of a pre-concentrator with a sensor can allow the measurement of very low levels of gas concentrations (e.g., below the detection limit) and can also improve the accuracy of the measurement of low levels of concentration by shifting the effective concentration seen by the sensor to a higher value, for which the measurement accuracy is better. According to one embodiment, FIG. 7C illustrates the gas sensor(s) 2052 with a first gas sensor 2053 and a second gas sensor with a gas pre-concentrator 2058.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered. Accordingly, the above description does not bind the scope of the claims.

### Example Computing System

Having described multiple aspects of sensor calibration, an example of a computing system in which various aspects of the disclosure may be implemented will now be described with respect to FIG. 10. According to one or more aspects, a computer system as illustrated in FIG. 10 may be incorporated as part of a computing device, which may implement, perform, and/or execute any and/or all of the features, methods, and/or method steps described herein. For example, computing device 2000 may represent some of the components of a hand-held device. A hand-held device may be any computing device with an input sensory unit, such as a camera and/or a display unit. Examples of a hand-held device include but are not limited to, video game consoles, tablets, smart phones, and mobile devices. In one embodiment, the computing device 2000 is configured to implement the computing devices described in FIGS. 1 and 2. FIG. 10 provides a schematic illustration of one embodiment of a computing device 2000 that can perform the methods provided by various other embodiments, as described herein, and/or can function as the host computer system, a remote kiosk/terminal, a point-of-sale device, a mobile device, a set-top box, and/or a computer system. FIG. 10 is meant only to provide a generalized illustration of various components, any and/or all of which may be utilized as appropriate. FIG. 10, therefore, broadly illustrates how individual system elements may be implemented in a relatively separated or relatively more integrated manner.

The computing device 2000 is shown comprising hardware elements that can be electrically coupled via a bus 2005 (or may otherwise be in communication, as appropriate). The hardware elements may include one or more processors 2010, including without limitation one or more general-purpose processors and/or one or more special-purpose processors (such as digital signal processing chips, graphics acceleration processors, and/or the like); one or more input devices 2015, which can include without limitation a camera, a mouse, a keyboard and/or the like; and one or more output devices 2020, which can include without limitation a display unit, a printer and/or the like. The bus 2005 may also couple different processor and/or different cores within a single or multiple processors. According to some embodiments, the processor(s) 2010 can include a calibration unit 2012. Additionally, according to some embodiments, the processor(s) 2010 can execute the embodiments described in FIG. 3 and FIGS. 5A-5F.

In addition, computing device 2000 may further include a plurality of sensors 2050, that may include, but are not limited by, one or more gas sensors (e.g., gas sensor 2052), one or more accelerometers (e.g., accelerometer 2055), one or more gyroscopes (e.g., gyroscope 2060), humidity sensor 2085, atmospheric pressure sensor 2090 and/or one or more magnetometers (e.g., magnetometer 2065), as also illustrated in FIG. 2. Accelerometer 2055 may measure and/or provide (e.g., to processor 2010) information about acceleration experienced by computing device 2000 and/or otherwise sensed by accelerometer 2055. Gyroscope 2060 may similarly measure and/or provide (e.g., to processor 2010) information about gyroscopic motion (e.g., changes in orientation) experienced by computing device 2000 and/or otherwise sensed by gyroscope 2060. In addition, magnetometer 2065 may measure and provide (e.g., to processor 2010) information regarding measurements of and/or changes in magnetic field (e.g., changes in bearing) experienced by computing device 2000 and/or otherwise sensed by magnetometer 2065. In some embodiments, filters may be implemented in one or more of the sensors discussed above, or implemented as separate hardware or software components, for example as stored in the operating system 2040, application 2045, or other portion of the working memory 2035 or the storage device 2025.

The computing device 2000 may further include (and/or be in communication with) one or more non-transitory storage devices 2025, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data storage, including without limitation, various file systems, database structures, and/or the like. In some instances, storage devices 2025 can include the software code/program to implement the embodiments described in FIG. 3 and FIGS. 5A-5F.

The computing device 2000 might also include a communications subsystem 2030, which can include without limitation a modem, a network card (wireless or wired), an infrared communication device, a wireless communication device and/or chipset (such as a Bluetooth® device, an 802.11 device, a WiFi device, a WiMax device, cellular communication facilities, etc.), and/or the like. The communications subsystem 2030 may permit data to be exchanged with a network (such as the network described below, to name one example), other computer systems, and/or any other devices described herein. In many embodiments, the computing device 2000 will further comprise a non-transitory working memory 2035, which can include a RAM or ROM device, as described above. In some embodiments, the positioning unit(s) 2080 is included in the communications subsystem 2030. In some embodiments, the positioning unit(s) 2080 is included in the processor 2010 and/or in the device 2015 and/or 2020.

The computing device 2000 also can comprise software elements, shown as being currently located within the working memory 2035, including an operating system 2040, device drivers, executable libraries, and/or other code, such as one or more application programs 2045, which may comprise computer programs provided by various embodiments, and/or may be designed to implement methods, and/or configure systems, provided by other embodiments, as described herein. Merely by way of example, one or more modules (e.g., calibration unit or module) described with respect to the method(s) discussed above, might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

As noted above, in one or more embodiments, the computing device 2000 may be configured to implement the computing device of FIG. 1 and FIG. 2 discussed previously. In these embodiments, hardware components described above with respect to FIG. 1 and FIG. 2 may correspond to similar components described here with respect to computing device 2000. Additionally or alternatively, components described above with respect to FIG. 1 and FIG. 2 that might not be described here with respect to computing device 2000 may be added to and/or otherwise incorporated with computing device 2000 (e.g., in a manner similar to how such components may be included in FIG. 1 and FIG. 2).

A set of these instructions and/or code might be stored on a computer-readable storage medium, such as the storage device(s) 2025 described above. In some cases, the storage medium might be incorporated within a computer system, such as computing device 2000. In other embodiments, the storage medium might be separate from a computer system (e.g., a removable medium, such as a compact disc), and/or provided in an installation package, such that the storage medium can be used to program, configure and/or adapt a general purpose computer with the instructions/code stored thereon. These instructions might take the form of executable code, which is executable by the computing device 2000 and/or might take the form of source and/or installable code, which, upon compilation and/or installation on the computing device 2000 (e.g., using any of a variety of generally available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

Some embodiments may employ a computer system (such as the computing device 2000) to perform methods in accordance with the disclosure. For example, some or all of the procedures of the described methods may be performed by the computing device 2000 in response to processor 2010 and/or calibration unit 2012 executing one or more sequences of one or more instructions (which might be incorporated into the operating system 2040 and/or other code, such as an application program 2045) contained in the working memory 2035. Such instructions may be read into the working memory 2035 from another computer-readable medium, such as one or more of the storage device(s) 2025. Merely by way of example, execution of the sequences of instructions contained in the working memory 2035 might cause the processor(s) 2010 and/or calibration unit 2012 to perform one or more procedures of the methods described herein.

The terms “machine-readable medium” and “computer-readable medium,” as used herein, refers to any medium that participates in providing data that causes a machine to operate in a specific fashion. In an embodiment implemented using the computing device 2000, various computer-readable media might be involved in providing instructions/code to processor(s) 2010 and/or calibration unit 2012 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 2025. Volatile media include, without limitation, dynamic memory, such as the working memory 2035. Transmission media include, without limitation, coaxial cables, copper wire and fiber optics, including the wires that comprise the bus 2005, as well as the various components of the communications subsystem 2030 (and/or the media by which the communications subsystem 2030 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of computer-readable media may be involved in carrying one or more sequences of one or more instructions to the processor(s) 2010 and/or calibration unit 2012 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computing device 2000. These signals, which might be in the form of electromagnetic signals, acoustic signals, optical signals and/or the like, are all examples of carrier waves on which instructions can be encoded, in accordance with various embodiments of the invention.

The communications subsystem 2030 (and/or components thereof) generally will receive the signals, and the bus 2005 then might carry the signals (and/or the data, instructions, etc. carried by the signals) to the working memory 2035, from which the processor(s) 2010 and/or calibration unit 2012 retrieves and executes the instructions. The instructions received by the working memory 2035 may optionally be stored on a non-transitory storage device 2025 either before or after execution by the processor(s) 2010 and/or calibration unit 2012.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

Specific details are given in the description to provide a thorough understanding of the embodiments. However, embodiments may be practiced without these specific details. For example, well-known circuits, processes, algorithms, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

Also, some embodiments were described as processes. Although each may describe the operations as a sequential process, many of the operations can be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

Having described several embodiments, various modifications, alternative constructions, and equivalents may be used without departing from the spirit of the disclosure. For example, the above elements may merely be a component of a larger system, wherein other rules may take precedence over or otherwise modify the application of the invention. Also, a number of steps may be undertaken before, during, or after the above elements are considered. Accordingly, the above description does not limit the scope of the disclosure.

What is claimed is:

1. A method for obtaining accurate gas sensor data, the method comprising:
receiving, by a mobile device, gas sensor data from at least one gas sensor, wherein the mobile device includes the at least one gas sensor and at least one environmental component;
receiving, by the mobile device, environmental data from the at least one environmental component; and
calibrating, by the mobile device, the received gas sensor data based on the received environmental data and one or more reference characteristic curves.

2. The method of claim 1, wherein the at least one gas sensor includes a Metal Oxide Semiconductor (MOS) gas sensor.

3. The method of claim 1, wherein the at least one gas sensor includes an electrochemical gas sensor.

4. The method of claim 1, wherein the at least one gas sensor includes a polymer-based sensor.

5. The method of claim 1, wherein the at least one gas sensor includes a spectroscopic absorption sensor.

6. The method of claim 1, wherein the at least one gas sensor is configured to sense carbon monoxide (CO), nitric oxide (NO), nitrogen dioxide (NO₂), ammonia (NH₃), or carbon dioxide (CO₂).

7. The method of claim 1, wherein the at least one environmental component includes a positioning unit, and wherein the method further comprising:
determining, using the positioning unit, that the mobile device is at an outdoor location;
receiving, using a communications subsystem, clean air data for the outdoor location, wherein the clean air data is associated with an air quality index that indicates a particular quality of air; and
calibrating the received gas sensor data associated with a zero reading based on the clean air data.

8. The method of claim 7, wherein the clean air data is received from an environmental agency or a weather station.

9. The method of claim 1, wherein the mobile device includes a communications subsystem, and wherein the one or more reference characteristic curves is received from the communications subsystem.

10. The method of claim 1, wherein the at least one environmental component includes a temperature sensor, and wherein the method further comprising:
determining a temperature characteristic curve for the gas sensor data associated with a plurality of temperatures, based on the environmental data from the temperature sensor; and
wherein the calibration of the received gas sensor data is based on the temperature characteristic curve.

11. The method of claim 1, wherein the at least one environmental component includes a humidity sensor, and wherein the method further comprising:
determining a humidity characteristic curve for the gas sensor data associated with a plurality of humidities, based on the environmental data from the humidity sensor; and
wherein the calibration of the received gas sensor data is based on the humidity characteristic curve.

12. The method of claim 1, wherein the at least one environmental component includes an atmospheric pressure sensor, and wherein the method further comprising:
determining an atmospheric pressure characteristic curve for the gas sensor data associated with a plurality of atmospheric pressures, based on the environmental data from the atmospheric pressure sensor; and
wherein the calibration of the received gas sensor data is based on the atmospheric pressure characteristic curve.

13. The method of claim 1, wherein the at least one environmental component includes a heater, and wherein the method further comprising:
heating up the at least one gas sensor by turning on the heater;
determining a heating up gas sensor resistance characteristic curve for the gas sensor data associated with a plurality of temperatures as the at least one gas sensor is heating up; and
wherein the calibration of the received gas sensor data is based on the heating up gas sensor resistance characteristic curve.

14. The method of claim 1, wherein the at least one environmental component includes a heater, and wherein the method further comprising:
cooling down the at least one gas sensor by turning off the heater;
determining a cooling down gas sensor resistance characteristic curve for the gas sensor data associated with a plurality of temperatures as the at least one gas sensor is cooling down; and
wherein the calibration of the received gas sensor data is based on the cooling down gas sensor resistance characteristic curve.

15. The method of claim 1, wherein the calibration is based on a sensor noise level associated with the at least one gas sensor.

16. The method of claim 1, wherein the at least one gas sensor includes a first gas sensor and a second gas sensor attached to a filter.

17. The method of claim 1, wherein the at least one gas sensor includes a first gas sensor and a second gas sensor attached to a gas pre-concentrator.

18. The method of claim 1, wherein the at least one gas sensor includes a first gas sensor and a second gas sensor with a different sensitivity to sensing carbon monoxide.

19. A mobile device for obtaining accurate gas sensor data comprising:
memory;
at least one gas sensor configured to generate gas sensor data;
at least one environmental component configured to generate environmental data; and
one or more processors configured to calibrate the generated gas sensor data based on the generated environmental data and one or more reference characteristic curves.

20. The mobile device of claim 19, wherein the at least one gas sensor includes a Metal Oxide Semiconductor (MOS) gas sensor.

21. The mobile device of claim 19, wherein the at least one gas sensor includes an electrochemical gas sensor.

22. The mobile device of claim 19, wherein the at least one gas sensor includes a polymer-based sensor.

23. The mobile device of claim 19, wherein the at least one gas sensor includes a spectroscopic absorption sensor.

24. The mobile device of claim 19, wherein the at least one gas sensor is configured to sense carbon monoxide (CO), nitric oxide (NO), nitrogen dioxide (NO₂), ammonia (NH₃), or carbon dioxide (CO₂).

25. The mobile device of claim 19, wherein the at least one gas sensor includes a first gas sensor and a second gas sensor attached to a filter.

26. The mobile device of claim 19, wherein the at least one gas sensor includes a first gas sensor and a second gas sensor attached to a gas pre-concentrator.

27. The mobile device of claim 19, wherein the at least one gas sensor includes a first gas sensor and a second gas sensor with a different sensitivity to sensing carbon monoxide.

28. One or more computer-readable media storing computer-executable instructions for obtaining accurate gas sensor data that, when executed, cause one or more computing devices included in a mobile device to:
receive gas sensor data from at least one gas sensor, wherein the mobile device includes the at least one gas sensor and at least one environmental component;
environmental data from the at least one environmental component; and
calibrate the received gas sensor data based on the received environmental data and one or more reference characteristic curves.

29. An apparatus for obtaining accurate gas sensor data comprising:
means for receiving gas sensor data from at least one gas sensor;
means for receiving environmental data from at least one sensor component; and
means for calibrating the received gas sensor data based on the received environmental data and one or more reference characteristic curves.

30. The apparatus of claim 29, further comprising:
means for determining that the mobile device is at an outdoor location based on positioning data;
means for receiving clean air data for the outdoor location, wherein the clean air data is associated with an air quality index that indicates a particular quality of air; and
means for calibrating the received sensor data associated with a zero reading based on the clean air data.
